(19) United States
(12) Patent Application Publication
(10) Pub. No.: US 2019/0231740 A1
(43) Pub. Date: Aug. 1, 2019

(54) BLOOD FLOW-IMPROVING AGENT, ROYAL JELLY COMPOSITION, AND METHOD FOR PRODUCING ROYAL JELLY COMPOSITION

(71) Applicant: YAMADA BEE COMPANY, INC., Okayama (JP)

(72) Inventors: Michiru KAN, Okayama (JP); Ayanori YAMAKI, Okayama (JP)

(73) Assignee: YAMADA BEE COMPANY, INC., Okayama (JP)

(21) Appl. No.: 16/312,209

(22) PCT Filed: Jun. 16, 2017
(86) PCT No.: PCT/JP2017/022390

§ 371 Date: Dec. 20, 2018

(30) Foreign Application Priority Data
Jun. 23, 2016 (JP) 2016-124464

(51) Int. Cl. A61K 31/355; A61K 35/644; A61P 7/00

(52) U.S. Cl. CPC A61K 31/355; A61K 31/455; A61P 7/00; A61K 35/644

(57) ABSTRACT

The present invention provides a blood flow-improving agent comprising 10-hydroxydecanoic acid as an active ingredient.

### TECHNICAL FIELD

The present invention relates to a blood flow-improving agent, a royal jelly composition, and a method for producing a royal jelly composition.

### BACKGROUND ART

As a herbal medicine having an effect to improve the blood circulation, for example, Toki-shakuyaku-san is known. In Patent Literature 1, a blood circulation-improving drug containing vitamin E and hepronicate that is a nicotinic acid-based peripheral vasodilator is disclosed. In Patent Literature 2, a peripheral blood flow-improving agent containing an extract of a plant of Brassicaceae Lepidium is disclosed.

### CITATION LIST

### Patent Literature

Patent Literature 1: JP 2003-119135 A

Patent Literature 2: JP 2005-281272 A

### SUMMARY OF INVENTION

### Technical Problem

Hepronicate that is used in the blood circulation-improving drug disclosed in Patent Literature 1 causes side effects on digestive organs, skins and nervous system, and therefore, this blood circulation-improving drug is unsuitable for routine use. The blood flow-improving agent described in Patent Literature 2 uses an extract of a plant such as maca, and a blood flow-improving agent that is more easily obtainable has been desired.

It is an object of the present invention to provide a novel blood flow-improving agent. It is another object of the present invention to provide a royal jelly composition having high blood flow-improving action.

### Solution to Problem

The present inventors have newly found that 10-hydroxydecanoic acid has particularly excellent blood flow-improving action.

The present invention provides a blood flow-improving agent comprising 10-hydroxydecanoic acid as an active ingredient. Since the blood flow-improving agent comprises 10-hydroxydecanoic acid as an active ingredient, it can improve blood flow.

The present invention also provides a food composition for improving blood flow, the composition comprising the blood flow-improving agent. By ingesting the food composition, blood flow can be improved.

The above-described present invention can also be said to be 10-hydroxydecanoic acid for use in the blood flow improvement. The present invention can also be considered to be an agent comprising 10-hydroxydecanoic acid as an active ingredient, the agent being for use in the blood flow improvement.

The present invention can also be considered to be use of 10-hydroxydecanoic acid in the manufacturing of a blood flow-improving agent.

The present invention can also be considered to be a blood flow-improving method comprising administering an effective amount of 10-hydroxydecanoic acid to the subject in need thereof.

The present invention also provides a royal jelly composition, the content of 10-hydroxydecanoic acid being more than 1.1 mass % in the solid content of the composition. The present invention also provides a royal jelly composition, the content ratio by mass of trans-10-hydroxy-2-decenoic acid to 10-hydroxydecanoic acid in the composition being less than 3.5. Since the royal jelly composition has increased content or content ratio of 10-hydroxydecanoic acid, the composition has high blood flow-improving action.

The present invention also provides a method for producing a royal jelly composition, comprising making the content of 10-hydroxydecanoic acid in the royal jelly composition be more than 1.1 mass % or making the content ratio by mass of trans-10-hydroxy-2-decenoic acid to 10-hydroxydecanoic acid in the royal jelly composition be less than 3.5. Through the production method, a royal jelly composition having high blood flow-improving action can be obtained.

### Advantageous Effects of Invention

A novel blood flow-improving agent is provided by the present invention. Furthermore, a royal jelly composition having high blood flow-improving action is provided by the present invention.

### BRIEF DESCRIPTION OF DRAWING

FIG. 1 is a graph showing a blood flow volume before administration of a test substance and after one hour from administration thereof.

### DESCRIPTION OF EMBODIMENTS

An embodiment of the present invention will be described in detail hereinafter. However, the present invention is not limited to the following embodiment.

The blood flow-improving agent of the present invention comprises 10-hydroxydecanoic acid as an active ingredient.

The 10-hydroxydecanoic acid is one of constituents of royal jelly. In the blood flow-improving agent according to the present embodiment, the 10-hydroxydecanoic acid may be a synthetic product, or may be a naturally occurring one such as royal jelly-derived one. The 10-hydroxydecanoic acid derived from royal jelly can be obtained by, for example, extraction from royal jelly with an organic solvent or the like and purification.

In the present specification, the blood flow improvement refers to an increase in blood flow volume. The blood flow-improving agent according to the present embodiment comprises 10-hydroxydecanoic acid as an active ingredient, and therefore, by ingesting it, the blood flow volume, particularly peripheral blood flow volume, can be increased. The blood flow-improving agent according to the present embodiment can also be said to be a peripheral blood flow volume-increasing agent.

The blood flow-improving agent according to the present embodiment can be used in a dose of, for example, 6 mg to 120 mg per day for an adult weighing 60 kg, and it is preferable to use it in a dose of 12 mg to 40 mg. The dosage can be appropriately determined in the above range according to factors, such as health condition of a person who ingests, administration method and combination with other agents.

The blood flow-improving agent according to the present embodiment may be orally administered (ingested), or may be parenterally administered. The mucosal immuno stimulator of the present invention may be administered once a day or may be divided and administered a plurality of times, such as twice a day or three times a day, as long as the amount of the active ingredient per day is in the aforesaid range.

The blood flow-improving agent according to the present embodiment may comprise 10-hydroxydecanoic acid in an amount of, for example, not less than 0.2 mass % or not less than 0.4 mass %, based on the total solid content of the blood flow-improving agent, and it is preferable that the blood flow-improving agent comprise not less than 0.5 mass % of the acid, and it is more preferable that the blood flow-improving agent comprise not less than 1.0 mass % of the acid. The 10-hydroxydecanoic acid content in the blood flow-improving agent may be not more than 50.0 mass %, or may be not more than 20.0 mass %, or may be not more than 10.0 mass %, based on the total solid content.

The blood flow-improving agent may comprise the above-described active ingredient only, or may further comprise other ingredients as long as they do not hinder the effects of the present invention. Examples of other ingredients include pharmaceutically acceptable ingredients (e.g., excipients, binders, lubricants, disintegrants, emulsifying agents, surfactant, bases, solubilizing agents, suspending agents), and ingredients acceptable as foods (e.g., minerals, vitamins, flavonoids, quinones, polyphenols, amino acids, nucleic acids, essential fatty acids, freshener, binders, sweeteners, disintegrants, lubricants, colorants, flavoring agents, stabilizing agents, preservatives, sustained-release adjusting agents, surface active agents, dissolving agents, wetting agents).

The blood flow-improving agent may be in any state of solids, liquids or pastes, and may be in any of dosage forms, such as tablets (including uncoated tablets, sugar-coated tablets, effervescent tablets, film-coated tablets, chewable tablets, troches, etc.), capsules, pills, powder (powdered drugs), fine granules, granules, liquids, suspensions, emulsions, syrups, pastes, and injections (including a case where when an injection is used, it is blended with distilled water or an infusion such as amino acid infusion or electrolyte infusion to prepare a liquid drugs). These various formulations can be prepared by, for example, mixing 10-hydroxydecanoic acid that is an active ingredient and other optional ingredients and shaping the mixture into the above dosage forms.

The blood flow-improving agent according to the present embodiment can be used as a medicine, a quasi-drug and a food composition itself, and can be used by adding it to a medicine, a quasi-drug and a food composition. It is preferable that the food composition be food emphasized on a third function of food (physical condition adjusting function). Examples of the foods emphasized on a third function of food include health foods, foods with function claims, dietary supplements, supplements and foods for specified health uses.

A medicine, a quasi-drug or a food composition consisting of the blood flow-improving agent according to the present embodiment, or a medicine, a quasi-drug or a food composition comprising the blood flow-improving agent according to the present embodiment may be one for improving blood flow, and may be accompanied by indications “to the effect that it improves blood flow”, “to the effect that it maintains healthy blood flow (peripheral blood flow)”, “to the effect that it increases blood flow (peripheral blood flow) volume”, “to the effect that it improves blood circulation”, “to the effect that it improves circulation of blood”, “to the effect that it ameliorates peripheral circulatory impairment”, etc. On these indications, conditions such as “under the cooling conditions” and “in decrease in atmospheric temperature or room temperature” may be set. Moreover, conditions of specific parts, such as “limbs”, “fingertips and toes”, “hands and feet”, “fingers”, “lower back”, “abdomen”, “upper back” and “shoulder”, may be set. Furthermore, the blood flow-improving agent according to the present embodiment can be used for ameliorating stiff neck. A medicine, a quasi-drug or a food composition consisting of the blood flow-improving agent according to the present embodiment, or a medicine, a quasi-drug or a food composition comprising the blood flow-improving agent according to the present embodiment may be accompanied by an indication “to the effect that it ameliorates stiff neck”, etc. On this indication, conditions such as “in the driving for a long time, deskwork or computer work” may be set.

The food composition comprising the blood flow-improving agent according to the present embodiment may comprise 10-hydroxydecanoic acid in an amount of, for example, 0.2 to 10.0 mass %. Examples of the food compositions include the following ones, and by mixing the blood flow-improving agent and intermediate products during the production process for them or final products, food compositions used for the above purposes can be obtained: beverages, e.g., coffee, soft drinks such as juice and tea beverage, milk-based drinks, lactic acid bacteria beverages, yogurt drinks, carbonated drinks, and liquors, such as sake, western liquor, fruit wine and honey wine; spreads, such as custard cream; pastes, such as fruit paste; western confectionery, such as chocolate, doughnut, pie, cream puff, gum, jelly, candy, cookie, cake and pudding; Japanese confectionery, such as rice cake stuffed with sweet red bean jam, rice cake, bean-paste bun, Japanese sponge cake, Anmitsu and sweet bean jelly; ice confectionery, such as ice cream, ice candy and sherbet; cooked foods, such as curry, beef bowl, rice porridge, miso soup, soup, meat sauce, pasta, pickles and jam; and seasonings, such as dressing, dried seasoning powder, flavor enhancer and soup stock.

The present invention described above can also be considered to be 10-hydroxydecanoic acid for use in the blood flow improvement. The present invention can also be considered to be an agent comprising 10-hydroxydecanoic acid as an active ingredient, the agent being for use in the blood flow improvement. The present invention can also be considered to be use of 10-hydroxydecanoic acid in the production of a blood flow-improving agent.

The present invention can also be considered to be a blood flow-improving method comprising administering an effective amount of 10-hydroxydecanoic acid to the subject in need thereof.

The present invention also provides a royal jelly composition, wherein the content of 10-hydroxydecanoic acid in the solid content of the composition is more than 1.1 mass %.

In natural royal jelly, water, protein, carbohydrate, lipid, ash, free amino acid, vitamin, mineral, etc. are contained, and as the lipids, 10-hydroxydecanoic acid, trans-10-hydroxy-2-decenoic acid and the like are inherently contained. In typical royal jelly from China, the content of the 10-hydroxydecanoic acid in the solid content is approximately not less than 0.4 mass % but not more than 1.1 mass %.

The content of the 10-hydroxydecanoic acid in the solid content of the royal jelly composition may be a content of more than 1.1 mass %, or may be not less than 1.2 mass % or not less than 1.5 mass %, and it is preferable that the content thereof be not less than 3.0 mass %, it is more preferable that the content thereof be not less than 3.3 mass %, and it is still more preferable that the content thereof be not less than 5.0 mass %. The content may be, for example, not more than 50 mass %, or may be not more than 10 mass %.

The content of the 10-hydroxydecanoic acid in the royal jelly composition can be increased by, for example, adding 10-hydroxydecanoic acid to the royal jelly, or can also be increased by converting trans-10-hydroxy-2-decenoic acid contained in the royal jelly into 10-hydroxydecanoic acid.

The present invention also provides a royal jelly composition in which the content ratio of trans-10-hydroxy-2-decenoic acid to 10-hydroxydecanoic acid is less than 3.5. In natural royal jelly in which the ratio has not been controlled, the above ratio is about 3.5 to 5.5. In the royal jelly composition according to the present embodiment, the above ratio may be not more than 3.4, may be not more than 3.0, may be not more than 1.4, or may be not more than 1.0. The above ratio may be 0. That is to say, the royal jelly composition may not comprise trans-10-hydroxy-2-decenoic acid. The royal jelly composition may comprise 10-hydroxydecanoic acid in an amount of not less than 1.0 mass %, may comprise the acid in an amount of more than 1.1 mass %, or not less than 1.2 mass %, or not less than 1.5 mass %, may comprise the acid in an amount of not less than 3.0 mass %, or may comprise the acid in an amount of not less than 3.3 mass %, based on the solid content. The royal jelly composition may comprise 10-hydroxydecanoic acid in an amount of, for example, not more than 50 mass %, or not more than 10 mass %, based on the solid content.

The content ratio of trans-10-hydroxy-2-decenoic acid to 10-hydroxydecanoic acid in the royal jelly composition can be decreased by, for example, adding 10-hydroxydecanoic acid to the royal jelly to increase the amount of 10-hydroxydecanoic acid, or can also be decreased by, for example, converting trans-10-hydroxy-2-decenoic acid contained in the royal jelly into 10-hydroxydecanoic acid.

The present invention also provides a method for producing a royal jelly composition, comprising making the content of 10-hydroxydecanoic acid in the royal jelly composition be more than 1.1 mass % or making the content ratio by mass of trans-10-hydroxy-2-decenoic acid to 10-hydroxydecanoic acid in the royal jelly composition be less than 3.5. The content of the 10-hydroxydecanoic acid in the resulting royal jelly composition may be more than 1.1 mass %, and it is preferable that the content thereof be not less than 1.5 mass %, it is more preferable that the content thereof be not less than 3.0 mass %, it is still more preferable that the content thereof be not less than 3.3 mass %, and it is much more preferable that the content thereof be not less than 5.0 mass % in terms of the solid content. Since the royal jelly composition obtained by the above production method has been increased in the 10-hydroxydecanoic acid content, it is excellent in blood flow-improving action. The content of the 10-hydroxydecanoic acid may be not more than 50 mass % or may be not more than 10 mass %, based on the total solid content of the royal jelly composition. In the resulting royal jelly composition, the content ratio by mass of trans-10-hydroxy-2-decenoic acid to 10-hydroxydecanoic acid may be less than 3.5, may be not more than 1.4, or may be not more than 1.0. The above ratio may be 0. That is to say, the royal jelly composition may not comprise trans-10-hydroxy-2-decenoic acid.

The step of making the mass content of 10-hydroxydecanoic acid in the royal jelly composition be more than 1.1 mass % or the step of making the content ratio by mass of trans-10-hydroxy-2-decenoic acid to 10-hydroxydecanoic acid in the royal jelly composition be less than 3.5 may specifically comprise, for example, a step of adding 10-hydroxydecanoic acid to the royal jelly, a step of converting trans-10-hydroxy-2-decenoic acid contained in the royal jelly into 10-hydroxydecanoic acid, or both of them.

In the present specification, the royal jelly may be, for example, raw royal jelly, or may be a royal jelly treated product prepared by subjecting raw royal jelly to treatment. The raw royal jelly can be obtained for example as a beekeeping product in accordance with a conventional method. Examples of the royal jelly treated products include a royal jelly concentrate or dilution product prepared by concentrating or diluting raw royal jelly, a royal jelly powder prepared by drying and powdering raw royal jelly, enzymatically hydrolyzed royal jelly prepared by treating raw royal jelly with a proteolytic enzyme, and a royal jelly organic solvent extract prepared by extracting raw royal jelly with an organic solvent such as ethanol, e.g., a royal jelly ethanol extract. The royal jelly treated product may be royal jelly having been subjected to a plurality of treatments. The royal jelly may be an enzymatically hydrolyzed royal jelly powder prepared by enzymatic hydrolysis and powdering. Moreover, royal jelly of any area of production, such as royal jelly produced in Japan, China, New Zealand or Europe, can be used. In particular, royal jelly produced in Japan or China is preferable from the viewpoint of ease of obtaining.

The royal jelly concentrate can be obtained by, for example, removing moisture content from raw royal jelly. The royal jelly dilution product can be obtained by, for example, adding water or the like to raw royal jelly.

The royal jelly powder can be obtained by, for example, powdering raw royal jelly by a known method in the present technical field, such as freeze drying or spray drying. After the freeze drying or the spray drying, milling may be carried out using a mill (e.g., pin mill, hammer mill, ball mill, jet mill) to obtain a royal jelly powder.

The enzymatically hydrolyzed royal jelly can be obtained by, for example, treating raw royal jelly with a proteolytic enzyme. Examples of the proteolytic enzymes include an enzyme having endopeptidase action, an enzyme having exopeptidase action, and an enzyme having both of endopeptidase action and exopeptidase action.

The royal jelly organic solvent extract can be obtained by, for example, extracting raw royal jelly using, as a solvent, an organic solvent such as ethanol, methanol, propanol or acetone. The extraction time can be appropriately set according to the form of the raw royal jelly used as a starting material, the type and the amount of the solvent, the temperature and the stirring conditions in the extraction, etc. After the extraction, a solid matter may be removed by filtration, centrifugation or the like. The extracted solution may be used as it is, or the solution may be used as a concentrated solution or a powder by removing the solvent from the solution. It is preferable that the royal jelly organic solvent extract be a royal jelly ethanol extract.

Examples of the method of converting trans-10-hydroxy-2-decenoic acid contained in the royal jelly into 10-hydroxydecanoic acid include a method of hydrogenating an unsaturated bond in the trans-10-hydroxy-2-decenoic acid to convert it into 10-hydroxydecanoic acid. The hydrogenation of the unsaturated bond can be carried out by using a known technique such as a technique of using a catalyst. The above conversion may be carried out by the use of a microorganism. The microorganism used for the conversion only needs to have ability to convert the trans-10-hydroxy-2-decenoic acid into 10-hydroxydecanoic acid, and examples thereof include lactic acid bacteria and yeast.

The royal jelly composition has been increased in the content of 10-hydroxydecanoic acid, and therefore, it is excellent in blood flow-improving action. On that account, the royal jelly composition can be a composition for improving blood flow. In particular, the royal jelly composition can increase a peripheral blood flow volume by being ingested. Accordingly, the royal jelly composition can be a composition for increasing a peripheral blood flow volume. The royal jelly composition is excellent in blood flow-improving action, and therefore, it can also be used for ameliorating stiff neck.

The royal jelly composition may also be used as a medicine, a quasi-drug or a food composition, or may be added to a medicine, a quasi-drug or a food composition. To the medicine, the quasi-drug and the food composition, their production methods, dosages, dosing forms and formulation forms, and other ingredients contained therein in the royal jelly composition, embodiments similar to those in the case of the aforesaid blood flow-improving agent can be applied.

### EXAMPLES

Hereinafter, the present invention will be more specifically described with reference to the examples. However, the present invention is in not limited to the following examples.

As a test animal, a 6 to 8 week-old male rat Slc: SD rat (Japan SLC, Inc.) was used. As test substances, 10-hydroxydecanoic acid (manufactured by Combi-Blocks Inc.), trans-10-hydroxy-2-decenoic acid (referred to as “10-H2DA” hereinafter, manufactured by HANGZOU EASTBIOPHARM CO., LTD.) and Toki-shakuyaku-san (manufactured by TSUMURA & CO.) were used. A 0.5% w/v aqueous solution of methyl cellulose 400 (Wako Pure Chemical Industries, Ltd.) was prepared, and in the aqueous solution, the test substances were each suspended, thereby preparing a 12 mg/mL solution of 10-hydroxydecanoic acid, a 42 mg/ml solution of 10-H2DA and a 375 mg/ml solution of Toki-shakuyaku-san. The test animals were divided into four groups each consisting of three animals, and these groups were referred to as a 10-hydroxydecanoic acid group, a 10-H2DA group, a Toki-shakuyaku-san group and a control group. Using a gastric tube, the respective methyl cellulose aqueous solutions were orally administered to the 10-hydroxydecanoic acid group in such a desired amount that the amount of 10-hydroxydecanoic acid was 120 mg/kg, to the 10-H2DA group in such a desired amount that the amount of 10-H2DA was 420 mg/kg, and to the Toki-shakuyaku-san group in such a desired amount that the amount of Toki-shakuyaku-san was 3750 mg/kg. To the control group, a control substance (0.5% w/v methyl cellulose aqueous solution 10 mL/kg) was orally administered using a gastric tube.

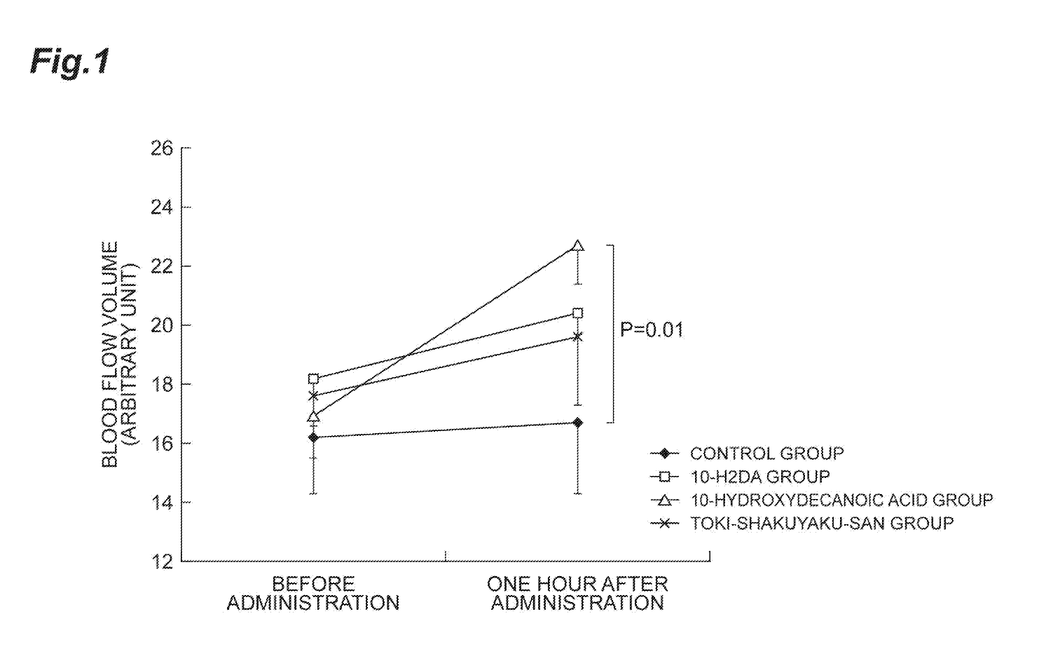

Before the administration of the test substance and after one hour from the administration, a blood flow volume of a hindlimb of the rat was measured in an awaking and non-fixed state. For the measurement, a two-dimensional laser blood flow meter OZ-1 (manufactured by OMEGAWAVE INC.) was used. The blood flow volume of each group is shown in FIG. 1. An increase in the blood flow volume (average value of three rats) based on that before the administration of each of the control group, the 10-hydroxydecanoic acid group, the 10-H2DA group and the Toki-shakuyaku-san group after one hour from the administration is shown in Table 1.

TABLE 1

| Substance administered | Increase in blood flow volume (arbitrary unit) |
|---|---|
| Control substance | 0.5 |
| 10-H2DA | 2.2 |
| 10-Hydroxydecanoic acid | 5.8 |
| Toki-shakuyaku-san | 2.0 |

With regard to the hindlimb blood flow volume, an apparent change between before and after the administration was not observed in the control group (FIG. 1, Table 1). In the 10-H2DA group and the Toki-shakuyaku-san group, a certain increase in the blood flow volume was confirmed, and in the 10-hydroxydecanoic acid group, a remarkable increase in the blood flow volume was observed (Table 1). After one hour from the administration, a significant difference (P=0.01) was observed between the control group and the 10-hydroxydecanoic acid group (FIG. 1).

1-2. (canceled)

3. A royal jelly composition, wherein the content of 10-hydroxydecanoic acid in the solid content of the composition is more than 1.1 mass %.

4. A royal jelly composition, wherein the content ratio by mass of trans-10-hydroxy-2-decenoic acid to 10-hydroxydecanoic acid in the composition is less than 3.5.

5. A method for producing the royal jelly composition according to claim 3, comprising
making the content of 10-hydroxydecanoic acid in the royal jelly composition be more than 1.1 mass %.

6. A method for producing the royal jelly composition according to claim 4, comprising making the content ratio by mass of trans-10-hydroxy-2-decenoic acid to 10-hydroxydecanoic acid in the royal jelly composition be less than 3.5.

7. A method for improving blood flow, the method comprising administering an effective amount of a blood flow-improving agent comprising 10-hydroxydecanoic acid to a subject in need thereof.

8. The method according to claim 7, wherein the blood flow-improving agent is administered in the form of food composition.
